(19) United States
(12) Patent Application Publication
(10) Pub. No.: US 2009/0171172 A1
(43) Pub. Date: Jul. 2, 2009

(54) METHOD AND SYSTEM FOR PULSE GATING

(75) Inventors: Daryl L. Bordon, Livermore, CA (US); Michael P. O'Neil, Pleasanton, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC, Boulder, CO (US)

(21) Appl. No.: 12/339,242

(22) Filed: Dec. 19, 2008

Related U.S. Application Data
(60) Provisional application No. 61/009,453, filed on Dec. 28, 2007.

(51) Int. Cl. A61B 5/02; A61B 5/00; A61B 5/1455

(52) U.S. Cl. 600/324; 600/483; 600/323

(57) ABSTRACT

The present disclosure relates to the acquisition and use of an arterial pulse signal that may be used to synchronize the measurement of other physiological characteristics. In one embodiment, a sensor is provided that emits light toward a pulsing artery and detects the transmitted light to generate a signal representative of the amount of light detected. In another embodiment, a sensor is provided that acquires physiological data from a first emitter and first detector placed proximate to a perfused tissue site and acquires arterial pulse data from a second emitter and second detector placed proximate to an artery. Embodiments related to systems, tangible media, and methods of operation are also provided.

### RELATED APPLICATION

This application claims priority from U.S. Patent Application No. 61/009,453 which was filed on Dec. 28, 2007, and is incorporated herein by reference in its entirety.

### BACKGROUND

The present disclosure relates generally to medical devices and, more particularly, to sensors used for sensing physiological parameters of a patient.

This section is intended to introduce the reader to various aspects of art that may be related to various aspects of disclosed embodiments, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices and techniques have been developed for monitoring physiological characteristics. Such devices and techniques provide doctors and other healthcare personnel with the information they need to provide the best possible healthcare for their patients. As a result, these monitoring devices and techniques have become an indispensable part of modern medicine.

Non-invasive medical devices may be particularly useful and desirable, as they generally provide immediate feedback and do not traumatize a patient. For example, certain types of non-invasive sensors transmit electromagnetic radiation, such as light, through a patient's tissue. Such sensors photoelectrically detect the absorption and/or scattering of the transmitted or reflected light in the tissue. The light emitted into the tissue is typically selected to be of one or more wavelengths that may be absorbed and scattered by particular tissue constituents under investigation. One or more physiological characteristics may then be calculated based upon the amount of light absorbed and/or scattered as the light passes through tissue.

For example, one such non-invasive technique for monitoring certain physiological characteristics of a patient is commonly referred to as pulse oximetry, and the devices built based upon pulse oximetry techniques are commonly referred to as pulse oximeters. Pulse oximetry may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time varying amount of arterial blood in the tissue during each cardiac cycle.

Pulse oximetry readings measure the pulsatile, dynamic changes in amount and type of blood constituents in tissue. However, events other than the pulsing of arterial blood, such as noise caused by patient motion, may lead to modulation of the light path, direction, and/or the amount of light detected by the sensor, introducing error to the measurements. As a result, pulse oximetry measurements that are performed in the presence of patient motion may suffer due to the arterial portion of the signal being overwhelmed, obscured or distorted by the portion of the signal attributable to the patient motion.

### SUMMARY

Certain aspects commensurate in scope with this disclosure are set forth below. It should be understood that these aspects are presented merely to provide the reader with a brief summary of certain forms any claimed invention might take and that these aspects are not intended to limit the scope of any claimed invention. Indeed, any claimed invention may encompass a variety of aspects that may not be set forth below.

According to an embodiment, there may be provided a sensor. The sensor may comprise an emitter configured to emit light toward a pulsing artery. The sensor may also comprise a detector configured to detect the transmitted or reflected light and to generate a signal representative of the amount of light detected.

According to an embodiment, there may be provided a sensor. The sensor may comprise a first emitter and a first detector configured to optically acquire physiological data when placed proximate to a perfused tissue site. The sensor may also comprise a second emitter and a second detector configured to optically acquire arterial pulse data when placed proximate to an artery.

According to an embodiment, there may be provided a monitoring system. The monitoring system may comprise a processor. The processor may be configured to process data representing a physiological characteristic of interest and data representing an arterial pulse. The processor may also be configured to generate a measure of the physiological characteristic of interest based upon the processed physiological characteristic and pulse data.

According to an embodiment, there may be provided a method for measuring a physiological characteristic. The method may includes the acts of acquiring data related to a physiological characteristic of interest and of acquiring data related to an arterial pulse. The arterial pulse may be derived from the data related to the arterial pulse. A measure of the physiological characteristic of interest may be derived using the data related to a physiological characteristic and the arterial pulse.

According to an embodiment, there may be provided one or more tangible media encoded with a processor-executable program. The program may comprise code for deriving an arterial pulse based upon data acquired from a sensor or part of a sensor placed proximate to an artery. The program may also comprise code for deriving a measure of a physiological characteristic of interest based upon data acquired related to the physiological characteristic and upon the arterial pulse.

### BRIEF DESCRIPTION OF THE DRAWINGS

Advantages of this disclosure may become apparent upon reading the following detailed description and upon reference to the drawings in which:

FIG. 1 illustrates a patient monitoring system coupled to a multi-parameter patient monitor and corresponding sensors, in accordance with aspects of an embodiment;

FIG. 2 is a block diagram of a monitoring system, in accordance with aspects of an embodiment;

FIG. 3 is a block diagram of a monitoring system, in accordance with aspects of a further embodiment;

FIG. 4 is a block diagram of a monitoring system, in accordance with aspects of an additional embodiment; and

FIG. 5 is a block diagram of a monitoring system, in accordance with aspects of another embodiment.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In accordance with the present disclosure, systems for pulse oximetry, or other applications utilizing spectrophotometry, may be provided that identify arterial pulses using optical or other techniques. In certain embodiments, this identification is performed utilizing data obtained from a sensor package configured to acquire the arterial pulse data. In certain embodiments, this sensor package may be separate from or part of an existing sensor package for measuring a physiological parameter, such as a pulse oximeter sensor.

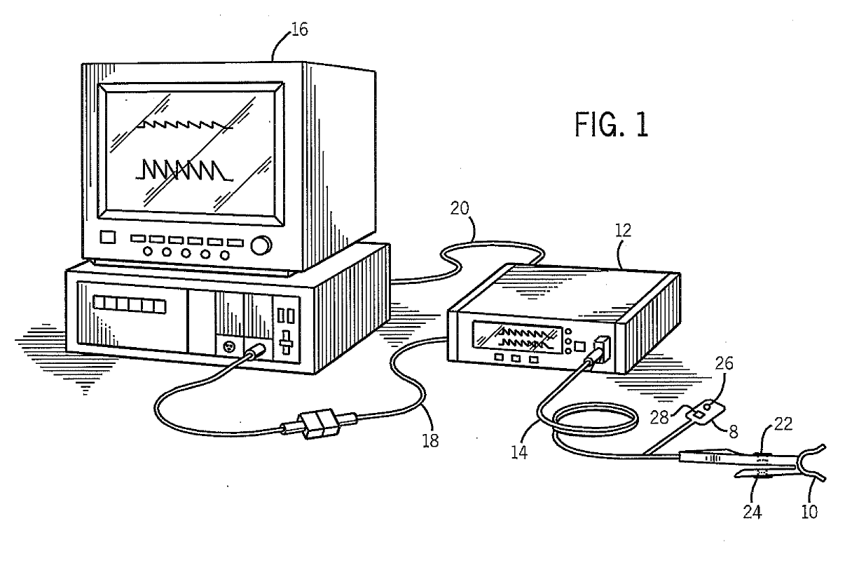

For example, referring now to FIG. 1, a pulse sensor 8 and physiological sensor 10 according to the present invention may be used in conjunction with a patient monitor 12. In the depicted embodiment, a cable 14 connects both the pulse sensor 8 and the physiological sensor 10 to the patient monitor 12. In other embodiments, the pulse sensor 8 and physiological sensor 10 may be separately connected to the patient monitor 12 by separate respective cables. Likewise, in other embodiments, the components of the pulse sensor 8 and the physiological sensor 10 may be provided in a common sensor package, i.e., as a combined sensor.

In certain embodiments, one or more of the sensors 8, 10 and/or the cable 14 may include or incorporate one or more integrated circuit devices or electrical devices, such as a memory, processor chip, or resistor, that may facilitate or enhance communication between the sensors 8, 10 and the patient monitor 12. Likewise the cable 14 may be an adaptor cable, with or without an integrated circuit or electrical device, for facilitating communication between the sensors 8, 10 and various types of monitors, including older or newer versions of the patient monitor 12 or other physiological monitors. In other embodiments, the sensors 8, 10 and the patient monitor 12 may communicate via wireless means, such as using radio, infrared, or optical signals. In such embodiments, a transmission device (not shown) may be connected to the sensors 8, 10 to facilitate wireless transmission between the sensors 8, 10 and the patient monitor 12. As will be appreciated by those of ordinary skill in the art, the cable 14 (or a corresponding wireless transmission) is typically used to transmit control or timing signals from the monitor 12 to the sensors 8, 10 and/or to transmit acquired data from the sensors 8, 10 to the monitor 12. In some embodiments, the cable 14 may be an optical fiber that enables optical signals to be conducted between the patient monitor 12 and the sensors 8, 10.

In one embodiment, the patient monitor 12 may be a suitable pulse oximeter, such as those available from Nellcor Puritan Bennett LLC and/or Covidien. In other embodiments, the patient monitor 12 may be a monitor suitable for measuring tissue water fractions, or other body fluid related metrics, using spectrophotometric or other techniques. Furthermore, the patient monitor 12 may be a multi-purpose monitor suitable for performing pulse oximetry and measurement of tissue water fraction, or other combinations of physiological and/or biochemical monitoring processes, using data acquired via the sensors 8, 10. Furthermore, to upgrade conventional monitoring functions provided by the monitor 12 and to provide additional functions, the patient monitor 12 may be coupled to a multi-parameter patient monitor 16 via a cable 18 connected to a sensor input port and/or a cable 20 connected to a digital communication port.

In the depicted embodiment, the physiological sensor 10 is configured as a transmission-type sensor and includes optical components, such as one or more emitters 22 and a detector 24, which may be of any suitable type. Likewise, in the depicted embodiment, the pulse sensor 8 is configured as a reflectance-type sensor and includes a respective emitter 26 and detector 28. In certain embodiments, one or more of the emitters 22, 26 may include light emitting diodes adapted to transmit one or more wavelengths of light, such as in the red to infrared range, and one or both of the detectors 24, 28 may be photodetectors, such as silicon photodiode packages, selected to receive light in the ranges emitted by the respective emitters 22, 26.

In the depicted embodiment, the pulse sensor 8 and physiological sensor 10 are jointly coupled to a cable 14 that is responsible for transmitting electrical and/or optical signals to and from the emitters 22, 26 and the detectors 24, 28. The cable 14 may be permanently coupled to one or both of the pulse sensor 8 and the physiological the sensor 10, or it may be removably coupled to one or both of these sensors—the latter alternative being more useful and cost efficient in situations where one or more of the sensors is disposable. Further, as noted above, in certain embodiments, the pulse sensor 8 and the physiological sensor 10 may have separate respective cables 14 such that the sensors are separately connectable to the monitor 12.

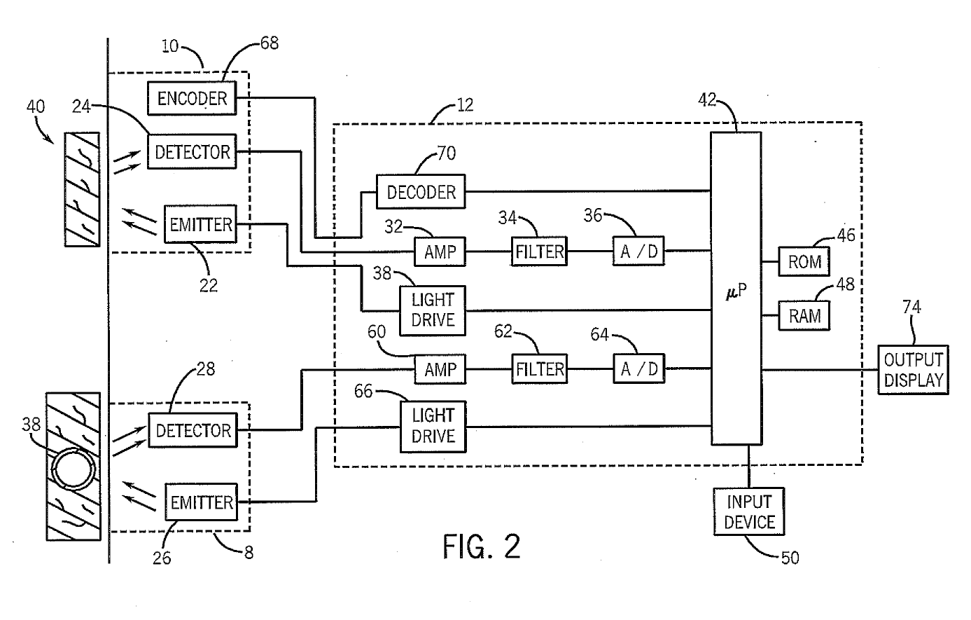

With the foregoing system description in mind, we refer now to FIG. 2 where an embodiment of a monitor 12, pulse sensor 8, and physiological sensor 10 are discussed. In particular, FIG. 2 illustrates a block diagram depicting a pulse sensor 8, physiological sensor 10, and monitor 12 for use in a monitoring system in accordance with an exemplary embodiment. As previously described the pulse sensor 8 and physiological sensor 10 respectively include one or more emitters 22, 24 as well as respective photodetectors 24, 28. In the depicted embodiment, the emitters 22, 26 of the respective pulse sensor 8 and physiological sensor 10 are configured to transmit electromagnetic radiation, such as light, into the tissue 40 of a patient.

In an embodiment where the monitoring system is a pulse oximetry system, the emitters 22 of the physiological sensor 10 may be configured to emit light at wavelengths that are differentially absorbed by oxygenated and deoxygenated hemoglobin, such as at a red and infrared wavelengths. For example, in such a pulse oximetry implementation, the emitter 22 may include two light emitting diodes (LEDs) where one LED emits light at a first wavelength where the absorption of HbO₂ differs from the absorption of reduced Hb. In this example, the second wavelength, i.e., the wavelength of light emitted by the second LED, may be a wavelength where the absorption of Hb and HbO₂ differs from those at the first wavelength. For example, LED wavelengths for measuring normal blood oxygenation levels typically include a red light emitted at approximately 660 nm and an infrared light emitted at approximately 900 nm. In one such an embodiment, the LEDs of the emitter 22 are activated alternately such that only one wavelength is being emitted and detected at a time.

In one embodiment, the physiological sensor 10 includes a detector 24 configured to detect the scattered and reflected light and to generate a corresponding electrical signal. Examples of such detectors 24 include one or more photodiodes configured to detect light at one or more of the emitted wavelengths of interest. For example, in an embodiment in which emitter 22, such as a pair of LEDs in an oximetry implementation, only emit light at the wavelengths of interest and in which the emissions alternate, i.e., only light at one wavelength is emitted, a single detector 24 may be provided as long as the detector 24 is configured to detect light at each wavelength of interest. In the depicted embodiment, the physiological sensor 10 is depicted as a reflectance-type sensor, i.e., the emitters 22 and detector 24 are provide on the same side of the tissue 40 and the detector 24 detects lights that enters and exits the same surface of the tissue 40, i.e., the light is reflected back by interactions with the tissue 40.

In one embodiment, the pulse sensor 8 includes an emitter 26, such as a single LED. For example, in one embodiment the emitter 26 of the pulse sensor 8 is a single LED emitting at an infrared wavelength, such as the aforementioned 900 nm, though other wavelengths in the near-infrared spectrum (750 nm to 2500 nm) or in the infrared spectrum in general may also be employed. The detector 28 of the pulse sensor 8 may be a photodiode or of the suitable detector of the wavelengths emitted by the emitter 26. In this depicted embodiment, the pulse sensor 8 is also depicted as being a reflectance-type sensor.

In an embodiment where the physiological sensor 10 is a pulse oximetry sensor, the physiological sensor 10 may be situated above blood perfused tissue, such as on a fingertip, toe, earlobe, or forehead of the patient. In such an embodiment, the pulse sensor 8 may be situated above a pulsing artery 38, such as the temporal artery of the head. Such a position over a pulsing artery is generally not suitable to acquire data for measuring blood oxygen saturation (SpO₂), i.e., such a site is generally not suitable for pulse oximetry. However, in such an embodiment, the plethysmographic signal acquired by the pulse sensor 8 placed over such a pulsing artery 38 may provide a strong signal that indicates arterial pulsation and this signal may be used to synchronize processing of the data acquired elsewhere using the physiological sensor 10, such as a pulse oximetry sensor.

As discussed herein, in embodiments where the components of the pulse sensor 8 and the physiological sensor 10 are provided as separate sensors, these separate sensors may be placed on different parts of the patient's body and need not be proximate to one another. For example, in embodiments where the pulse sensor 8 and the physiological sensor 10 are separate, the physiological sensor 10 may be placed on the finger of the patient while the pulse sensor 8 is placed on the temporal artery or another pulsing artery 38 that may or may not be proximate to the location of the physiological sensor 10.

While the preceding examples disclose the use of optical techniques for acquiring arterial pulse data via the pulse sensor 8, in other embodiments other types of techniques may be employed to acquire the arterial pulse data. For example, in other embodiments the pulse sensor 8 may measure arterial pressure (such as via accelerometers or other pressure sensitive instrumentation) as an indication of arterial pulse. Likewise, in yet another embodiment, the pulse sensor 8 may measure impedance or other electrical indicia as an indicator of arterial pulse. In one other embodiment, the pulse sensor 8 may utilize acoustical data (such as via a microphone placed proximate the heart or a major artery) to detect arterial pulses.

In an exemplary embodiment, the pulse sensor 8 and the physiological sensor 10 provide their respective detected signals to a monitor 12. In this embodiment, the monitor 12 may have a microprocessor 42 that calculates a physiological parameter (such as blood oxygen saturation (SpO₂) in one example) based on the data provided by the physiological sensor 10 and the pulse sensor 8. In such an embodiment, the microprocessor 42 may be connected to other component pails of the monitor 12, such as a ROM 46, a RAM 48, and input device(s) 50. In one embodiment, the ROM 46 holds the algorithms used to process the measured signals and the RAM 48 stores the detected signal values or data for use in the algorithms.

In one embodiment, input device 50 allows a user to interface with the monitor 12, such as via buttons of an operator interface, a keypad or keyboard, or a mouse or other selection mechanism for use with a provided control interface. For example, a user may input or select parameters specific to the patient undergoing monitoring or may specify a monitor protocol where multiple protocols are available. For example, different wavelengths or wavelength combinations and/or different light emission timing schemes or measurement cycle lengths may be utilized in different protocols. As a result, different protocols may be desirable depending on the placement of the physiological sensor 10 and/or the pulse sensor 8.

As noted above, in certain embodiments detected signals are passed from the pulse sensor 8 and the physiological sensor 10 to the monitor 12 for processing. In the depicted embodiment, the signals are amplified and filtered in the monitor 12 by respective amplifiers 32, 60 and filters 34, 62 respectively, before being converted to digital signals by an analog-to-digital converters 36, 64, respectively. The signals may then be used to determine an arterial pulse and a blood oxygen saturation (or other physiological parameter) based on the arterial pulse. The monitor 12 may be configured to display the calculated parameters, such as the measured blood oxygen saturation based on the detected arterial pulses, on display 74.

In one embodiment, light drive units 38, 66 in the monitor 12 control the timing of one or more of the emitters 22, 26, respectively. While the depicted embodiment discloses the use of a separate light drive 66, amplifier 60, filter 62, and analog-to-digital converter for the pulse sensor 8, in other embodiments one or more of these components may support both the pulse sensor 8 and the physiological sensor 10. In other words, in other embodiments, there may be only one light drive, amplifier, filter, and/or analog-to-digital converter that supports both the pulse sensor 8 and the physiological sensor 10.

The depicted embodiment also includes an encoder 68 provided in at least the physiological sensor 10. Such an embodiment may be desirable where the emitter 22 (such as two LEDs in a pulse oximetry implementation) is manufactured to operate at one or more certain wavelengths and where variances in the wavelengths actually emitted by the emitter 22 may occur which may result in inaccurate readings. To help avoid inaccurate readings, an encoder 68 and decoder 70 may be used to calibrate the monitor 12 to the actual wavelengths being generated. The encoder 68 may be a resistor, for example, whose value corresponds to coefficients stored in the monitor 12. The coefficients may then be used in the processing algorithms. Alternatively, the encoder 68 may also be a memory device, such as an EPROM, that stores information, such as the coefficients themselves. Once the coefficients are determined by the monitor 12, they may be utilized to calibrate the monitor 12. Though the encoder 68 is depicted in the physiological sensor 10, the encoder may, alternatively, be provided in a cable 14 in embodiments in which the physiological sensor 10 and cable 14 are not separable. Further, an encoder as described herein may also be utilized in the pulse sensor 8 to provide calibration information to the monitor 12 for use in the calculation of arterial pulses by the microprocessor 42.

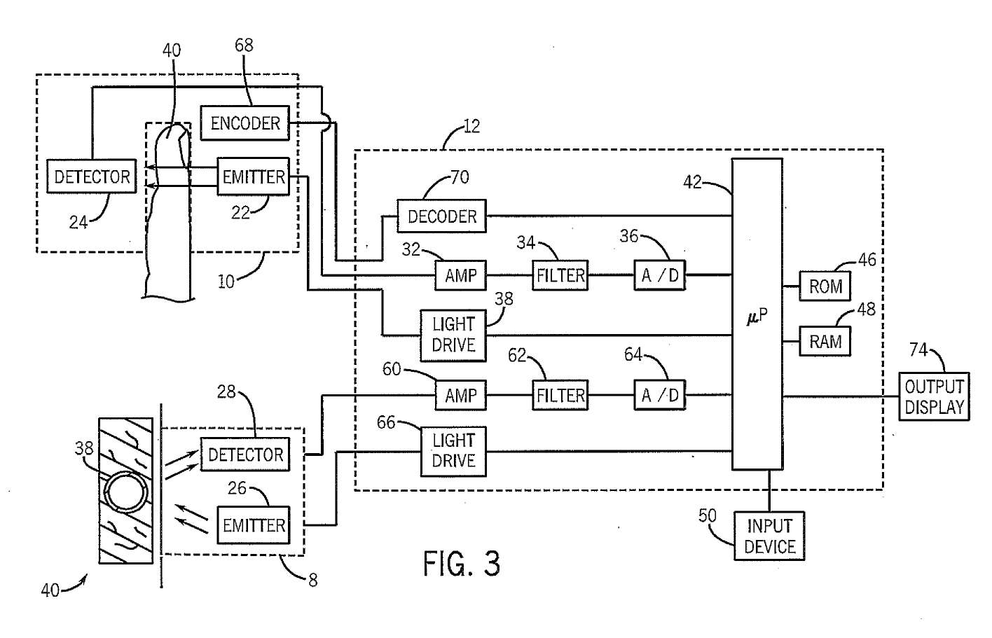

Turning now to FIG. 3, an embodiment is depicted where the physiological sensor 10 is configured as a transmission-type sensor. In this embodiment, the physiological sensor 10 may be configured to emit light from the emitter 22 through the tissue 40, such as the tissue of the finger or earlobe, toward the detector 24 positioned opposite the emitter 22 with respect to the tissue 40. Thus, in this embodiment, the detector 24 detects the light that has passed through the tissue as opposed to the light reflected by the tissue. Such an embodiment may be suitable for use where the pulse sensor 8 is utilized on or near tissue that is better suited for reflectance-type sensing, such as above the temporal artery or other suitable arteries, but where the physiological sensor 10 is utilized on or near tissue that is suitable for transmission-type sensing, such as fingers or earlobes.

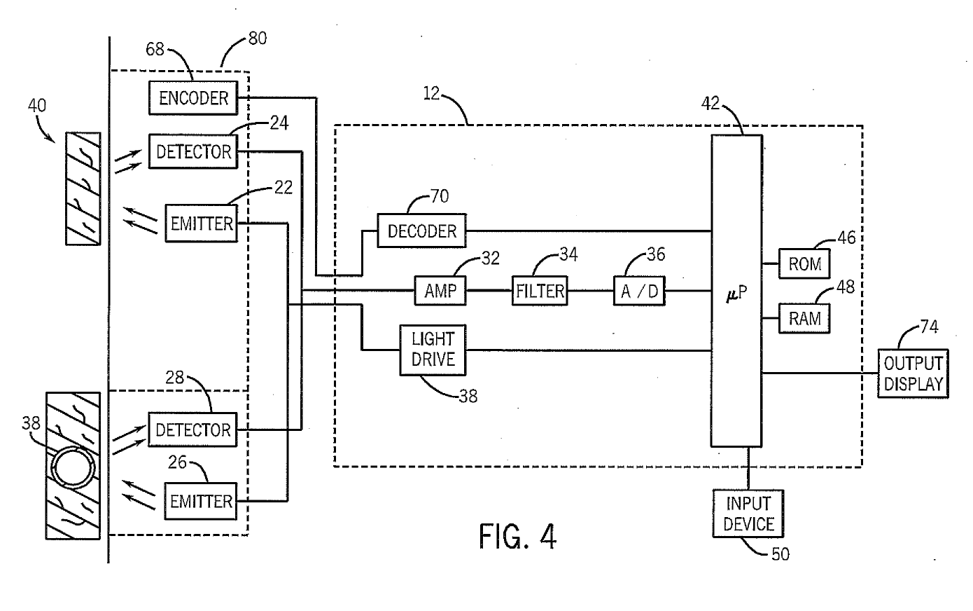

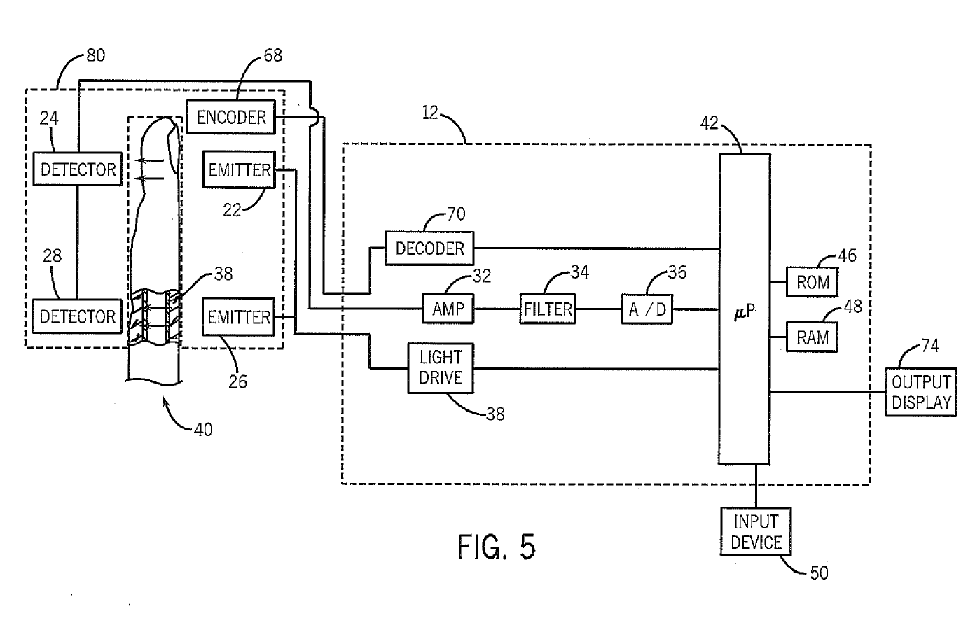

Unlike FIGS. 2 and 3 which depict separate pulse and physiological sensors, FIGS. 4 and 5 depict embodiments where there is a common sensor package 80 that includes both the emitter 22 and detector 24 used for sensing the physiological characteristic of interest as well as the emitter 26 and detector 28 used for sensing arterial pulses. For example, the embodiment depicted in FIG. 4 includes a common sensor package 80 configured as a reflectance-type sensor. In this embodiment, a single sensor package is provided that is configured such that the emitter 22 and detector 24 used for sensing a physiological signal of interest can be positioned suitably, such as over perfused tissue in an oximetry implementation. In addition, the single sensor package is configured such that the emitter 26 and detector 28 used to detect arterial pulse can be positioned over a suitable artery 30. Such an embodiment may be suitable for placement on the head of a patient such that the emitter 26 and detector 28 may be positioned over the temporal artery while the emitter 22 and detector 24 may be positioned over the perfused tissue of the forehead. Indeed, the common sensor package 80 may be configured to facilitate alignment and/or positioning of the respective emitters and detectors in such an implementation.

Likewise, FIG. 4 depicts an embodiment in which the common sensor package 80 is configured as a transmission-type sensor, with the emitter 22 and detector 24 situated opposite one another with respect to the perfused tissue 40. Similarly, the emitter 26 and detector 28 may be situated opposite one another with respect to a suitable artery 38. While the embodiments of FIGS. 4 and 5 generally depict a common sensor package 80 in which the optical components are all configured for reflectance or transmission-type sensing, in other embodiments the common sensor package 80 may be provided for a combination of transmission and reflectance-type sensing. For example, in one embodiment suitable for use on the hand, the emitter 22 and detector 24 for sensing the physiological trait of interest may be configured for transmission-type sensing, such as for placement on a finger tip. In this embodiment, the emitter 26 and detector 28 may be configured for reflectance type sensing, such as on the top or palm of the hand.

Thus in view of these embodiments, one or more sensor configurations may be provided that utilize optical or other data to provide a monitor with arterial pulse data that may be used to improve determination of a physiological trait, such as blood oxygen saturation levels. Such embodiments may be useful, for example, in the presence of patient motion where it is desirable to more readily identify those portions of a signal that correspond to an arterial pulse, as opposed to the motion component of the signal.

While any claimed invention may be susceptible to various modifications and alternative forms, specific embodiments have been shown by way of example in the drawings and have been described in detail herein. However, it should be understood that any claimed invention is not intended to be limited to the particular forms disclosed. Rather, the invention is to cover all modifications, equivalents, and alternatives falling within the spirit and scope of the invention as defined by the following appended claims.

What is claimed is:

1. A sensor comprising:
a first emitter and a first detector configured to optically acquire physiological data when placed proximate to a perfused tissue site; and
a second emitter and a second detector configured to optically acquire arterial pulse data when placed proximate to an artery.

2. The sensor of claim 1, wherein the first emitter comprises two or more LEDs configured to emit red and/or infrared wavelengths.

3. The sensor of claim 1, wherein the second emitter comprises an LED configured to emit at an infrared wavelength.

4. The sensor of claim 1, further comprising a common housing in which the first emitter, first detector, second emitter, and second detector are capable of being housed.

5. The sensor of claim 1, comprising:
a first housing in which the first emitter and first detector are capable of being housed;
a second housing in which second emitter and second detector are capable of being housed; and
a cable configured to couple both the first housing and the second housing to a monitor.

6. A monitoring system comprising:
a processor configured to process data representing a physiological characteristic of interest and data representing an arterial pulse, and to generate a measure of the physiological characteristic of interest based at least in part upon the processed physiological characteristic and pulse data.

7. The monitoring system of claim 6, further comprising:
a first sensor configured to acquire the data representing the physiological characteristic of interest; and
a second sensor configured to acquire the data representing the arterial pulse.

8. The monitoring system of claim 6, further comprising a sensor configured to acquire the data representing the physiological characteristic of interest using a first set of optical components, and to acquire the data representing the arterial pulse using a second set of optical components.

9. The monitoring system of claim 6, comprising a sensor configured to acquire the data representing the arterial pulse using absorbance or reflection of emitted light, changes in detected arterial pressure, changes in impedance associated with an artery, and/or acoustic indications of heart beat, and/or combinations thereof.

10. The monitoring system of claim 6, wherein the data representing the physiological characteristic of interest is acquired at least in part by a first sensor placed proximate to perfused tissue, and wherein the data representing the arterial pulse is acquired at least in part by a second sensor placed proximate to an artery.

11. The monitoring system of claim 6, further comprising one or more memory devices configured to store the data and/or to store routines for processing the data.

12. The monitoring system of claim 6, further comprising an output device configured to display the measure of the physiological characteristic of interest.

13. A method for measuring a physiological characteristic, comprising:
acquiring data related to a physiological characteristic of interest;
acquiring data related to an arterial pulse;
deriving the arterial pulse from the data related to the arterial pulse; and
deriving a measure of the physiological characteristic of interest based at least in part upon the data related to a physiological characteristic and the arterial pulse.

14. The method of claim 13, wherein acquiring data related to the physiological characteristic of interest comprises acquiring pulse oximetry data at a perfused tissue site.

15. The method of claim 13, wherein acquiring data related to the arterial pulse comprises acquiring absorbance or reflectance data for an infrared wavelength at site above an artery.

16. The method of claim 13, wherein acquiring data related to the arterial pulse comprises acquiring pressure, impedance, and/or acoustic data generally indicative of arterial pulsations.

17. The method of claim 13, wherein the data related to the arterial pulse comprises a plethysmographic signal generated using a single LED emitting infrared light over a pulsing artery.
